(19) United States
(12) Patent Application Publication
(10) Pub. No.: US 2007/0020623 A1
(43) Pub. Date: Jan. 25, 2007

(54) Method for determining homeostasis of the skin

(76) Inventors: Dirk Petersohn, Koeln (DE); Marcus Conradt, Koeln (DE); Kay Hoffmann, Koeln (DE)

Correspondence Address: WOODCOCK WASHBURN LLP, ONE LIBERTY PLACE, 46TH FLOOR, PHILADELPHIA, PA, 19103, US

(21) Appl. No.: 10/250,691

(22) PCT Filed: Dec. 20, 2001
(86) PCT No.: PCT/EP01/15179

§ 371 Date: Jan. 16, 2004

(30) Foreign Application Priority Data
Jan. 3, 2001 (DE) 101 00 127.4

(51) Int. Cl. C12Q 1/68

(52) U.S. Cl. 435/6

(57) ABSTRACT

Described herein are processes and devices useful for the identification of genes differentially expressed in skin. Preferably, the processes and devices are useful for the in vitro determination of skin homeostasis. Also described is a combination having a plurality of genes differentially expressed in skin compared to other tissues. Biochips and test kits useful in processes for the identification of genes expressed in skin homeostasis as well as in processes for screening substances useful in maintaining or promoting homeostasis of the skin are also described. Also described are processes for identifying substances useful in maintaining or promoting homeostasis of the skin as well as processes for making cosmetic and/or pharmaceutical preparations comprising such substances.

This invention relates to a process for the in vitro determination of the homeostasis of the skin in human beings or animals, to test kits and biochips for determining the homeostasis of the skin and to the use of proteins, mRNA molecules or fragments of proteins or mRNA molecules as markers for the homeostasis of the skin; also to a test for demonstrating the effectiveness of cosmetic or pharmaceutical active substances for maintaining or promoting the homeostasis of the skin or for the treatment of pathological conditions of the skin and to a screening process for identifying cosmetic or pharmaceutical active substances for maintaining or promoting, the homeostasis of the skin or for the treatment of pathological conditions of the skin and to a process for the production of a cosmetic or pharmaceutical preparation for maintaining or promoting the homeostasis of the skin or for the treatment of pathological conditions of the skin.

Aside from vegetative proliferation, the development of eukaryotic life begins with the fusion of two gametes. A zygote—the origin of each cell of a eukaryote—is formed. The time- and space-ordered differentiation of the daughter cells of a zygote is critical to the ontogenesis of a multicell organism. It leads to a variety of cell types differing in their morphology and their function. If, for example, the nerve cell of a human being is compared with a cell of the epidermis, the cells are found to be very different although both have the same origin and the same genome. The differentiation of cells goes along with modification of gene expression patterns. In the differentiated state, cells express the genes typical of them. Which genes play a role in the morphologies and functions of the skin cells, for example, has hitherto remained largely unclear. The ordered regulation of gene expression in the skin is crucially important to maintenance of the homeostasis of the organ. Each living cell is capable of reacting to signals from its environment. The reactions of the cells are achieved through the ordered regulation of gene expression so that the metabolism of cells is not static but very dynamic.

The expression of the genes in differentiated cells of the skin is not static, but very dynamic. Extracellular stimuli act on the transcription of living cells through partly complex signal transduction cascades. The regulation of transcription as an answer to extracellular signals is known as stimulus transcription coupling. Influencing this sensitive regulation mechanism can result in disturbance of the homeostasis of the skin and possibly in the formation and manifestation of pathogenic skin conditions.

According to the most recent estimates, the human genome comprises ca. 140,000 genes. However, of this immense supply of information, each cell uses only a small part specific to it for the synthesis of proteins which is reflected in the gene expression pattern. Which genes particularly in the skin—play a role has hitherto been largely unclear.

The skin is the largest organ of the human body. It is an organ of very complex structure which consists of a large number of different cell types and which forms the interface between the body and the environment. It follows from this that the cells of the skin are particularly exposed to exogenous physical and chemical environmental signals. The analysis of gene expression in the skin is crucially important to understanding reactions of the skin to exogenous stimuli.

A key feature of the skin is that, with increasing age, under the effect of skin-damaging stimuli or in cases of pathological skin conditions, the cells lose their ability to maintain the homeostasis of the organ. Which molecular mechanisms are behind this development has hitherto been largely unclear. The identification of new skin-specific markers makes it possible to understand the complex state of homeostasis, the formation and manifestation of pathogenic skin conditions. Only with this knowledge can new concepts be developed for skin treatment products.

Each cell type of the skin expresses ca. 15,000 different genes and synthesizes a corresponding number of proteins therefrom. However, which of these genes play a role in the homeostasis of the skin or are involved in pathogenic processes has hitherto been largely unclear.

The skin consists of several different cell types (fibroblasts, keratinocytes in various states of differentiation, melanocytes, Merkel cells, Langerhans cells, hair follicle cells, sweat gland cells, etc.) so that the complexity of genes expressed in the skin is immense. It has not yet been possible to describe this immense complexity. Nor has it yet been possible to identify from this complexity those genes which are expressed exclusively or particularly strongly in the skin.

In living cells, mRNA molecules occur in concentrations between just a few and several hundred copies. Hitherto, the weakly expressed genes have only been accessible to analyses with great difficulty, if at all. However, these molecules can play a crucial role in the homeostasis of the skin or can be involved in the formation or manifestation of pathogenic processes in the skin.

The totality of all the mRNA molecules which are synthesized at a certain time by a cell or a tissue is known as a “transcriptome”. Hitherto, it has not been possible to describe the complete transcriptome, i.e. the totality of all transcribed genes, of the human skin.

Although gene expression can be analyzed by quantification of specific mRNA molecules (for example northern blot, RNase protection experiments), only a relatively limited number of genes can be measured by these techniques.

Accordingly, there is a need to identify as many as possible and preferably all of the genes that are active in human or animal skin.

Accordingly, the problem addressed by the present invention was to identify as large a number of the genes expressed in human or animal skin as possible; to identify the genes of importance to the homeostasis of the skin and to provide processes for determining, the homeostasis of the skin by means of the identified genes.

According to the invention, the solution to this first problem is provided by a process (1) for the in vitro identification of the genes expressed in skin in human beings or animals which is characterized in that
a) a mixture of genetically coded factors expressed, i.e. transcribed, in human or animal skin, i.e. a mixture of mRNA molecules or fragments of mRNA molecules, is isolated from human or animal skin and
b) the mixture isolated in a) is subjected to a serial analysis of gene expression (SAGE) so that the genes expressed in human or animal skin are identified and their expression quantified.

According to the invention, the solution to the second problem is provided by a process (2) for the in vitro identification of the genes relevant to the homeostasis of the skin in: human beings or animals which is characterized in that
a) a mixture of genetically coded factors expressed, i.e. transcribed, in human or animal skin, i.e. a mixture of mRNA molecules or fragments of mRNA molecules, is isolated from human or animal skin and
b) the mixture isolated in a) is subjected to a serial analysis of gene expression (SAGE) so that the genes expressed in human or animal skin are identified and their expression quantified and
c) the analysis results from b) are compared with expression patterns of other tissues to identify the genes which are expressed to different extents (differentially) in skin and other tissues.

Expression patterns of other tissues can be accessed on-line in the data banks of the Cancer Genome Anatomy Project (CGAP) at the following address: http://cgap.nci.nih.gov/

The technique of “serial analysis of gene expression” (SAGE™) was used to determine the transcriptome of the skin. This technique enables all the genes expressed in the skin to be simultaneously identified and quantified. By comparing the transcriptome of the skin with the transcriptome of other tissues, it is possible to differentiate between relevant and non-relevant genes for the homeostasis of the skin.

Human skin from healthy female donors was used for the SAGE™ analysis. The SAGE™ analysis was carried out as described in EP-A-0 761 822 and in Velculescu, V. E. et al., 1995, Science 270, 484-487 and led to the identification of the genes active in skin. These genes are suitable for determining the homeostasis of the skin or for detecting pathological processes or conditions.

Table 6 contains a detailed list of the genes active in human skin as determined by process (1) according to the invention, indicating
consecutive order numbers in column 1,
the tag sequence used in column 2,
the relative expression frequency determined in skin in column 3,
the significance in column 4,
the UniGene Accession Number in column 5 and
a brief description of the gene or gene product in column 6.

Tables 1 to 5 contain a detailed list of the genes differentially expressed in skin and in other tissues as determined by process (2) according to the invention, indicating
consecutive order numbers in column 1,
the tag sequence used in column 2,
the relative expression frequency determined in the CGAP (Cancer Genome Anatomy Project) in column 3,
the relative expression frequency determined in skin in column 4,
the quotient of the frequencies (from column 3 and column 4) in column 5,
the significance in column 6,
the UniGene Accession Number in column 7 and
a brief description of the gene or gene product in column 8.

The quotient in column 5 indicates the strength of the differential expression, i.e. the factor by which the particular gene is expressed more strongly in the skin than in other tissues.

Table 7 contains a list of the genes expressed differentially by a factor of 13.33 to 211.11, indicating
consecutive order numbers in column 1,
the tag sequence used in column 2,
the relative expression frequency determined in the CGAP (Cancer Genome Anatomy Project) in column 3,
the relative expression frequency determined in skin in column 4,
the quotient of the frequencies (from column 3 and column 4) in column 5,
the UniGene Accession Number in column 6 and
a brief description of the gene or gene product in column 7.

The assignment of the tags to the genes defined by their Unigene Accession Number in column 6 was done by manual annotation.

The following data banks were used for the annotation:
1. Unigene—30.10.01 version with the following data bank entries:
   a. known genes from GenBank (12.10.01)
   b. ESTs from dbEST (19.10.01)
2. mRNA-version released on 17.10.01

The data banks were downloaded from the NCBI, formatted for a local version of the BLAST program (also NCBI) and compared for identical hits with the tags detected in the SAGE analysis.

The genes/clones found were checked for redundancy and finished as indicated below:
1. tag sequences with several different hits: evaluation as non-annotatable
2. tag sequences with double or several identical hits: elimination of the hits situated furthest away from the poly-A-tail.

The results from the Unigene databank were evaluated first and then compared with the results from the mRNA databank. The latter do not appear in Table 7 because they can also be called off via the Unigene entries.

All the links shown in the Results Table were tested on the 30.10.2001 database documented in the following (Unigene databank release: UniGene Build # 143):

Sequences Included in Unigene

Known genes are from GenBank (Oct. 12, 2001)

ESTs are from dbEST through Oct. 19, 2001

69367 mRNAs +gene CDSs

1147828 EST, 3′reads

1196006 EST, 5′reads

+598081 EST, other/unknown

------------

3011282 total sequences in clusters

Final Number of Clusters (sets)

96332 sets total

20516 sets contain at least one known gene

95171 sets contain at least one EST

19355 sets contain both genes and ESTs

Release Notes

The particular genes or gene products are disclosed under their UniGene Accession Number in the databank of the National: Center for Biotechnology Information (NCBI). This databank is accessible on-line at the following address: http://www.ncbi.nim.nih.gov/.

In addition, the genes or gene products are directly accessible at the following internet addresses:

http://www.ncbi.nim.nih.gov/UniGene/Hs.Home.html or

http://www.ncbi.nlm.nih.gov/genome/quide.

The data of the Cancer Genome Anatomy Project are accessible on-line at the following address: http://cgap.nci.nih.gov/

All genes which are expressed differentially by a factor of at least 2 and less than 5 are listed in Table 1.

All genes which are expressed differentially by a factor of at least 5 and less than 10 are listed in Table 2.

All genes which are expressed differentially by a factor of at least 10 and less than 20 are listed in Table 3.

All genes which are expressed differentially by a factor of at least 20 and less than 100 are listed in Table 4.

All genes which are expressed differentially by a factor of at least 100 are listed in Table 5.

According to the invention, the solution to the third problem addressed by the present invention is provided by a process (3) for the in vitro determination of the homeostasis of the skin in human beings or animals, more particularly in females, which is characterized in that
a) a mixture of proteins, mRNA molecules or fragments of proteins or mRNA molecules is isolated from human or animal skin,
b) the mixture isolated is tested for the presence and optionally the quantity of at least one of the proteins, mRNA molecules or fragments of proteins or mRNA molecules identified as expressed to different extents (differentially) in skin and other tissues by serial analysis of gene expression (SAGE),
c) the test results from b) are compared with the expression patterns identified by serial analysis of gene expression (SAGE) and
d) the mixture tested in b) is assigned to healthy skin or skin in homeostasis if it predominantly contains proteins, mRNA molecules or fragments of proteins or mRNA molecules which are expressed more strongly in skin than in other tissues or the mixture tested in b) is assigned to diseased skin or skin in disturbed homeostasis if it predominantly contains proteins, mRNA molecules or fragments of proteins or mRNA molecules which are expressed more strongly in other tissues than in skin.

It may be sufficient in step b) of the process for determining the homeostasis of the skin to test the isolated mixture for the presence of at least one of the proteins, mRNA molecules or fragments of proteins or mRNA molecules identified as differentially expressed in skin and other tissues by serial analysis of gene expression (SAGE) if they are expressed solely in skin or solely in other tissues. In all other cases, the quantity of differentially expressed molecules must also be determined, i.e. the expression must be quantified, in step b).

In step d) of the process for determining homeostasis of the skin, the mixture tested in b) is assigned to healthy skin or skin in homeostasis if it predominantly contains proteins, mRNA molecules or fragments of proteins or mRNA molecules which are expressed more strongly in skin than in other tissues, i.e. the mixture either contains more different compounds typically expressed in skin than those which are typically expressed in other tissues (qualitative differentiation) or more copies of compounds typically expressed in skin than typically present in other tissues (quantitative differentiation). A complementary procedure is adopted for assignment to diseased skin or skin in disturbed homeostasis.

A preferred embodiment of the process according to the invention for determining the homeostasis of the skin is characterized in that, in step b), the mixture isolated is tested for the presence and optionally the quantity of at least one of the proteins, mRNA molecules or fragments of proteins or mRNA molecules defined by their UniGene Accession Number in Tables 1 to 5 and 7, column 7, and in Table 7, column 6; in step c), the test results from b) are compared with the relative expression frequencies shown in Tables 1 to 5, columns 3 and 4 and the expression quotients indicated in column 5; and in step d), the mixture tested in b) is assigned to healthy skin or skin in homeostasis if it predominantly contains proteins, mRNA molecules or fragments of proteins or mRNA molecules which are expressed at least twice as strongly in skin as in other tissues or the mixture tested in b) is assigned to diseased skin or skin in disturbed homeostasis if it predominantly contains proteins, mRNA molecules or fragments of proteins or mRNA molecules which are expressed at least twice as strongly in other tissues as in skin.

Another preferred embodiment of the process according to the invention for determining the homeostasis of the skin is characterized in that, in step b), the mixture isolated is tested for the presence and optionally the quantity of at least one of the proteins, mRNA molecules or fragments of proteins or mRNA molecules defined by their UniGene Accession Number in Tables 2 to 5, column 7, and in Table 7, column 6; in step c), the test results from b) are compared with the relative expression frequencies shown in Tables 2 to 5, columns 3 and 4, and the expression quotients indicated in column 5; and in step d), the mixture tested in b) is assigned to healthy skin or skin in homeostasis if it predominantly contains proteins, mRNA molecules or fragments of proteins or mRNA molecules which are expressed at least five times as strongly in skin as in other tissues or the mixture tested in b) is assigned to diseased skin or skin in disturbed homeostasis if it predominantly contains proteins, mRNA molecules or fragments of proteins or mRNA molecules which are expressed at least five times as strongly in other tissues as in skin.

Another preferred embodiment of the process according to the invention for determining the homeostasis of the skin is characterized in that, in step b), the mixture isolated is tested for the presence and optionally the quantity of at least one of the proteins, mRNA molecules or fragments of proteins or mRNA molecules defined by their UniGene Accession Number in Tables 3 to 5, column 7, and in Table 7, column 6; in step c), the test results from b) are compared with the relative expression frequencies shown in Tables 3 to 5, columns 3 and 4, and the expression quotients indicated in column 5; and in step d), the mixture tested in b) is assigned to healthy skin or skin in homeostasis if it predominantly contains proteins, mRNA molecules or fragments of proteins or mRNA molecules which are expressed at least ten times as strongly in skin as in other tissues or the mixture tested in b) is assigned to diseased skin or skin in disturbed homeostasis if it predominantly contains proteins, mRNA molecules or fragments of proteins or mRNA molecules which are expressed at least ten times as strongly in other tissues as in skin.

Another preferred embodiment of the process according to the invention for determining the homeostasis of the skin is characterized in that, in step b), the mixture isolated is tested for the presence and optionally the quantity of at least one of the proteins, mRNA molecules or fragments of proteins or mRNA molecules defined by their UniGene Accession Number in Tables 4 and 5, column 7; in step c), the test results from b) are compared with the relative expression frequencies shown in Tables 4 and 5, columns 3 and 4, and the expression quotients indicated in column 5; and in step d), the mixture tested in b) is assigned to healthy skin or skin in homeostasis if it predominantly contains proteins, mRNA molecules or fragments of proteins or mRNA molecules which are expressed at least twenty times as strongly in skin as in other tissues or the mixture tested in b) is assigned to diseased skin or skin in disturbed homeostasis if it predominantly contains proteins, mRNA molecules or fragments of proteins or mRNA molecules which are expressed at least twenty times as strongly in other tissues as in skin.

Another preferred embodiment of the process according to the invention for determining the homeostasis of the skin is characterized in that, in step b), the mixture isolated is tested for the presence and optionally the quantity of at least one of the proteins, mRNA molecules or fragments of proteins or rRNA molecules defined by their UniGene Accession Number in Table 5, column 7; in step c), the test results from b) are compared with the relative expression frequencies shown in Table 5, columns 3 and 4, and the expression quotients indicated in column 5; and in step d), the mixture tested in b) is assigned to healthy skin or skin in homeostasis if it predominantly contains proteins, mRNA molecules or fragments of proteins or mRNA molecules which are expressed at least one hundred times as strongly in skin as in other tissues or the mixture tested in b) is assigned to diseased skin or skin in disturbed homeostasis if it predominantly contains proteins, mRNA molecules or fragments of proteins or mRNA molecules which are expressed at least one hundred times as strongly in other tissues as in skin.

The condition of the skin may also be described by quantifying several markers (expression products of the genes important to the homeostasis of the skin) which then have to be active in a certain ratio to one another in order to represent skin in homeostasis. Any deviations from that ratio point to the fact that the skin under analysis is not in homeostasis.

Accordingly, the present invention also relates to a process (4) for the in vitro determination of the homeostasis of the skin in human beings or animals, more particularly in females, which is characterized n that
a) a mixture of proteins, mRNA molecules or fragments of proteins or mRNA molecules is isolated from human or animal skin,
b) in the mixture isolated, at least two of the proteins, mRNA molecules or fragments of proteins or mRNA molecules identified as important to the homeostasis of the skin by process (2) are quantified,
c) the expression ratios of the at least two proteins, mRNA molecules or fragments of proteins or mRNA molecules to one another are determined,
d) the expression ratios from c) are compared with the expression ratios typically present in homeostatic skin for the molecules quantified in b), more particularly with the expression ratios shown in Table 6, column 3 and in Tables 1 to 5, column 4 and
e) the mixture isolated in a) is assigned to healthy skin or to skin in homeostasis if the expression ratios of the skin under analysis correspond to the expression ratios of skin in homeostasis or the mixture isolated in a) is assigned to diseased skin or to skin in disturbed homeostasis if the expression ratios of the skin under analysis differ from the expression ratios of skin in homeostasis.

In step a) of the process according to the invention for determining the homeostasis of the skin, the mixture is preferably isolated from a skin sample, more particularly from a whole skin sample or from an epidermis sample. The whole skin sample offers more comprehensive possibilities for comparison with the SAGE libraries which are similarly obtained from whole skin. By contrast, the epidermis sample is easier to obtain, for example by applying an adhesive plaster to the skin and stripping it off, as described in WO 00/10579 to the whole of which reference is hereby made.

In another embodiment of the process according to the invention for determining the homeostasis of the skin, the mixture is isolated in step a) by microdialysis. The technique of microdialysis is described, for example, in “Microdialysis: A method for measurement of local tissue metabolism”, Nielsen, P. S., Winge, K., Petersen, L. M.; Ugeskr Laeger 1999, Mar. 22, 161:12 1735-8: and in “Cutaneous microdialysis for human in vivo dermal absorption studies”, Anderson, C. et al.; Drugs Pharm. Sci., 1998, 91, 231-244; and also on-line at http://www.microdialysis.se/techniqu.htm, to the whole of which reference is hereby made. In microdialysis, a probe is typically inserted into the skin and slowly rinsed with a suitable carrier solution. After the acute reactions have abated after insertion, microdialysis yields proteins, mRNA molecules or fragments of proteins or mRNA molecules which occur in the extracellular space and which can then be isolated in vitro, for example by fractionation of the carrier liquid, and analyzed. Microdialysis is less invasive than the removal of a whole skin sample but has the disadvantage that it is limited to the isolation of compounds occurring in the extracellular space.

Another preferred embodiment of the process according to the invention for determining the homeostasis of the skin is characterized in that, in step b) in process (3), testing for the presence and optionally the quantity of at least one of the proteins or protein fragments; or, in process (4), the quantification of at least two proteins or protein fragments is carried out by a method selected from
one- or two-dimensional gel electrophoresis
affinity chromatography
protein/protein complexing in solution
mass spectrometry, more particularly Matrix Assisted Laser Desorption Ionization (MALDI) and, more particularly,
the use of protein chips,
   or suitable combinations of these methods.

These methods suitable for use in accordance with the invention are described in the overview by Akhilesh Pandey and Matthias Mann: “Proteomics to study genes and genomes”, Nature, Volume 405, Number 6788, 837-846 (2000) and the references cited therein, to the whole of which reference is hereby made.

2D gel electrophoresis is described, for example, in L. D. Adams, Two-dimensional Gel Electrophoresis using the Isodalt System or in L. D. Adams and S. R. Gallagher, Two-dimensional Gel Electrophoresis using the O'Farrell System; both in Current Protocols in Molecular Biology (1997), Eds. F. M. Ausubel et al.), Unit 10.3.1-10.4.13; or in 2-D Electrophoresis Manual; T. Berkelman, T. Senstedt; Amersham Pharmacia Biotech, 1998 (Order No. 80-6429-60).

The mass spectrometric characterization of the proteins or protein fragments is conducted in known manner, for example as described in the following literature references:

Methods in Molecular Biology, 1999; Vol. 112; 2-D Proteome Analysis Protocols; Editor: A. J. Link; Humana Press; Totowa; N. J.; cf. in particular Courchesne, P. L. and Patterson, S. D.; pp. 487-512.

Carr S. A. and Annan, R. S.; 1997; in: Current Protocols in Molecular Biology; Editor: Ausubel, F. M. et al.; John Wiley and Sons, Inc. 10.2.1-10.21.27.

Another preferred embodiment of the process according to the invention for determining the homeostasis of the skin is characterized in that, in step b) in process (3), testing for the presence and optionally the quantity of at least one of the mRNA molecules or mRNA molecule fragments; or, in process (4), the quantification of at least two mRNA molecules or mRNA molecule fragments is carried out by a method selected from

i. northern blots,

ii. reverse transcriptase polymerase chain reaction (RT-PCR),

iii. RNase protection experiments,

iv. dot blots,

v. cDNA sequencing,

vi. clone hybridization,

vii. differential display,

viii. subtractive hybridization,

ix. cDNA fragment finterprinting,

x. total gene expression analysis (TOGA)

xi. serial analysis of gene expression (SAGE) and, more particularly,

xii. the use of nucleic acid chips

or suitable combinations of these methods.

These methods suitable for use in accordance with the invention are described in the overviews by Akhilesh Pandey and Matthias Mann: “Proteomics to study genes and genomes”, Nature, Volume 405, Number 6788, 837-846 (2000) and “Genomics, gene expression and DNA arrays”, Nature, Volume 405, Number 6788, 827-836 (2000) and the references cited therein, to the whole of which reference is hereby made.

The TOGA process is described in “J. Gregor Sutcliffe et al., TOGA: An automated parsing technology for analyzing expression of nearly all genes, Proceedings of the National Academy of Sciences of the United States of America (PNAS), Vol. 97, No. 5, pp. 1976-1981 (2000)”, to the whole of which reference is hereby made.

According to the invention, however, other methods known to the expert may also be used to test for the presence and optionally the quantity of at least one of the proteins, mRNA molecules or fragments of proteins or mRNA molecules.

Another preferred embodiment of the process according to the invention for determining the homeostasis of the skin is characterized in that step b) comprises testing for the presence and optionally the quantity of 1 to about 5,000, preferably 1 to about 1,000, more preferably about 10 to about 500, most preferably about 10 to about 250, more particularly about 10 to about 100 and most particularly about 10 to about 50 of the proteins, mRNA molecules or fragments of proteins or mRNA molecules which are defined by their Unigene Accession Number in Tables 1 to 5, column 7, and in Table 7, column 6.

The present invention also relates to a test kit for the in vitro determination of the homeostasis of the skin in human beings or animals comprising means for carrying out the process according to the invention for determining the homeostasis of the skin.

The present invention also relates to a biochip for the in vitro determination of the homeostasis of the skin in human beings or animals comprising
i. a firm, i.e. rigid, or flexible support and,
ii. immobilized thereon, probes which are capable of binding specifically to at least one of the proteins, mRNA molecules or fragments of proteins or mRNA molecules which are defined by their UniGene Accession Number in Tables 1 to 5, column 7, and in Table 7, column 6.

A biochip is a miniaturized functional element with molecules, more particularly biomolecules, immobilized on a surface which are capable of acting as specific interaction partners. The structure of these functional elements often comprise rows and columns. They are then known as chip arrays. Since thousands of biological or biochemical functional elements can be arranged on one chip, they generally have to be made by microtechnical methods. Biological and biochemical functional elements include in particular DNA, RNA, PNA (in the case of nucleic acids and chemical derivatives thereof, single strands, triplex structures or combinations thereof, for example, may be present), saccharides, peptides, proteins (for example antibodies, antigens, receptors) and derivatives of combinatorial chemistry (for example organic molecules). Biochips generally have a two-dimensional base for coating with biologically or biochemically active materials. The bases may also be formed, for example, by walls of one or more capillaries or by channels. The prior art is represented, for example, by the following publications: Nature Genetics, Vol. 21, Supplement (entire), January 1999 (Biochips); Nature Biotechnology, Vol. 16, pp. 981-983, October 1998 (Biochips); Trends in Biotechnology, Vol. 16, pp. 301-306, July 1998 (Biochips) and the already cited overviews by Akhilesh Pandey and Matthias Mann: “Proteomics to study genes and genomes”, Nature, Volume 405, Number 6788, 837-846 (2000) and “Genomics, gene expression and DNA arrays”, Nature, Volume 405, Number 6788, 827-836 (2000) and the references cited therein, to the whole of which reference is hereby made.

A synoptic portrayal of the practical methods of using DNA chip technology can be found in the books “DNA Microarrays: A Practical Approach” (Editor: Mark Schena, 1999, Oxford University Press) and “Microarray Biochip Technology” (Editor: Mark Schena, 2000, Eaton Publishing), to the whole of which reference is hereby made.

The particularly preferred DNA chip technology in the context of the present invention is based on the ability of nucleic acids to enter into complementary base pairings. This technical principle known as hybridization has been used for years in southern blot and northern blot analysis. By comparison with these conventional methods where only a few genes are analyzed, DNA chip technology enables a few hundred to several thousand genes to be analyzed at the same time. A DNA chip consists essentially of a support material (for example glass or plastic) on which single-stranded gene-specific probes are immobilized in high density at a particular spot. The technique of probe application and the chemistry of probe immobilization are rated as problematical.

In the present state of the art, probe immobilization can be carried out in several ways:

E. M. Southern (E. M. Southern et al. (1992), Nucleic Acid Research 20, 1679-1684 and E. M. Southern et al. (1997), Nucleic Acid Research 25, 1155-1161) describes the production of oligonucleotide arrangements by direct synthesis on a glass surface derivatized with 3-glycidoxypropyl trimethoxysilane and then with a glycol. A similar process achieves the in situ synthesis of oligonucleotides by photosensitive combinatorial chemistry which may be compared with photolithographic techniques (Pease, A. C. et al. (1994), Proc. Natl. Acad. Sci. USA 91, 5022-5026).

Besides these techniques based on the in situ synthesis of oligonucleotides, already present DNA molecules can also be immobilized on surfaces of support material.

P. O. Brown (DeRisi et al. (1997), Science 278, 680-686) describes the immobilization of DNA on glass surfaces coated with polylysine.

The Article by L. M. Smith (Guo, Z. et al. (1994), Nucleic Acid Research 22, 5456-5465) discloses a similar process: oligonucleotides carrying a 5′-terminal amino group can be immobilized on a glass surface treated with 3-aminopropyl trimethoxysilane and then with 1,4-phenyl diisothiocyanate.

The DNA probes may be applied to a support using a so-called pin spotter. To this end, thin metal needles, for example 250 μm in diameter, dip into probe solutions and then transfer the adhering sample material in defined volumes to the support material of the DNA chip.

However, the probes are preferably applied by means of a piezo-controlled nanodispenser which—similarly to an ink jet printer—applies probe solutions With a volume of 100 picoliters to the surface of the support material without any contact.

The probes are immobilized as described, for example, in EP-A-0 965 647. DNA probes are generated by PCR using a sequence-specific primer pair, one primer being modified at the 5′-end and carrying a linker with a free amino group. This ensures that a defined strand of the PCR products can be immobilized on a glass surface treated with 3-aminopropyl trimethoxysilane and then with 1,4-phenyl diisothiocyanate. Ideally, the gene-specific PCR products should comprise a defined nucleic acid sequence with a length of 200400 bp and non-redundant sequences. After immobilization of the PCR products via the derivatized primer, the counter-strand of the PCR product is removed by incubation for 10 mins. at 96° C.

In one application typical of DNA chips, mRNA is isolated from two cell populations to be compared. The isolated mRNAs are converted into cDNA by reverse transcription using, for example, fluorescence-marked nucleotides. The samples to be compared are marked, for example, with red- or green-fluorescing nucleotides. The cDNAs are then hybridized with the gene probes immobilized on the DNA chip and the fixed fluorescences are then quantified.

The biochip according to the invention preferably comprises 1 to about 5,000, preferably 1 to about 1,000, more preferably about 10 to about 500, most preferably about 10 to about 250, more particularly about 10 to about 100 and most particularly about 10 to about 50 different probes. The different probes may be present as multiple copies on the chip.

The biochip according to the invention preferably comprises nucleic acid probes, more particularly RNA or PNA probes and most particularly DNA probes. The nucleic acid probes preferably have a length of about 10 to about 1,000, more preferably a length of about 10 to about 800, most preferably a length of about 100 to about 600 and, in one most particularly preferred embodiment, a length of about 200 to about 400 nucleotides.

In another preferred embodiment, the biochip according to the invention comprises peptide or protein probes, more particularly antibodies.

The present invention also relates to the use of the proteins, mRNA molecules or fragments of proteins or mRNA molecules which are defined by their UniGene Accession Number in Tables 1 to 5, column 7, and in Table 7, column 6, as markers for the homeostasis of the skin in human beings or animals.

The present invention also relates to a test for demonstrating the effectiveness of cosmetic or pharmaceutical active substances for maintaining or promoting the homeostasis of the skin or for the in vitro treatment of pathological conditions of the skin, such as neurodermatitis, sunburn, psoriasis, scleroderma, ichtyosis, atopic dermatitis, acne, seborrhoea, lupus erythematodes, roseacea, melanoma, basalioma, skin carcinoma, skin sarcoma, characterized in that
a) the status of the skin is determined by a process according to the invention for determining the homeostasis of the skin or with the aid of a test kit according to the invention for determining the homeostasis of the skin or with the aid of a biochip according to the invention,
b) an active substance for maintaining or promoting the homeostasis of the skin or for treating pathological skin conditions is applied one or more times to the skin,
c) the status of the skin is re-determined by a process according to the invention for determining, the homeostasis of the skin or with the aid of a test kit according to the invention for determining the homeostasis of the skin or with the aid of a biochip according to the invention and
d) the effectiveness of the active substance is determined by comparing the results from a) and c).

In order to accelerate the test, various active substances or placebos may be simultaneously applied to different areas of the skin. For example, an active substance may be applied to the left forearm and a placebo to the right forearm or vice versa.

The present invention also relates to a test kit for demonstrating the effectiveness of cosmetic or pharmaceutical active substances for maintaining or promoting the homeostasis of the skin or for the in vitro treatment of pathological skin conditions comprising means for carrying out the test according to the invention.

The present invention also relates to the use of the proteins, mRNA molecules or fragments of proteins or mRNA molecules which are defined by their UniGene Accession Number in Tables 1 to 5, column 7, and in Table 7, column 6, for demonstrating the effectiveness of cosmetic or pharmaceutical active substances for maintaining or promoting the homeostasis of the skin or for treating pathological skin conditions, such as neurodermatitis, sunburn, psoriasis, scleroderma, ichtyosis, atopic dermatitis, acne, seborrhoea, lupus erythematodes, roseacea, melanoma, basalioma, skin carcinoma, skin sarcoma.

The present invention also relates to a screening process for identifying cosmetic or pharmaceutical active substances for maintaining or promoting the homeostasis of the skin or for the in vitro treatment of pathological conditions of the skin, such as neurodermatitis, sunburn, psoriasis, scleroderma, ichtyosis, atopic dermatitis, acne, seborrhoea, lupus erythematodes, roseacea, melanoma, basalioma, skin carcinoma, skin sarcoma, characterized in that
a) the status of the skin is determined by a process according to the invention for determining the homeostasis of the skin or with the aid of a test kit according to the invention for determining the homeostasis of the skin or with the aid of a biochip according to the invention,
b) an active substance for maintaining or promoting the homeostasis of the skin or for treating pathological skin conditions is applied one or more times to the skin,
c) the status of the skin is re-determined by a process according to the invention for determining the homeostasis of the skin or with the aid of a test kit according to the invention for determining the homeostasis of the skin or with the aid of a biochip according to the invention and
d) effective active substances are determined by comparing the results from a) and c).

The present invention also relates to the use of the proteins, mRNA molecules or fragments of proteins or mRNA molecules which are defined by their UniGene Accession Number in Tables 1 to 5, column 7, and in Table 7, column 6, for identifying cosmetic or pharmaceutical active substances for maintaining or promoting the homeostasis of the skin or for treating of pathological skin conditions, such as neurodermatitis, sunburn, psoriasis, scleroderma, ichtyosis, atopic dermatitis, acne, seborrhoea, lupus erythematodes, roseacea, melanoma, basalioma, skin carcinoma, skin sarcoma.

The present invention also relates to a process for the production of a cosmetic or pharmaceutical preparation for maintaining or promoting the homeostasis of the skin or for treating pathological skin conditions, such as neurodermatitis, sunburn, psoriasis, scleroderma, ichtyosis, atopic dermatitis, acne, seborrhoea, lupus erythematodes, roseacea, melanoma, basalioma, skin carcinoma, skin sarcoma, characterized in that
a) effective active substances are determined by the screening process according to the invention or by the use for identifying cosmetic or pharmaceutical active substances for maintaining or promoting the homeostasis of the skin or for treating pathological skin conditions and
b) active substances found to be effective are mixed with cosmetically and pharmacologically suitable and compatible carriers.

Tables:

LENGTHY TABLE REFERENCED HERE
US20070020623A1-20070125-T00001
Please refer to the end of the specification for access instructions.

LENGTHY TABLE REFERENCED HERE
US20070020623A1-20070125-T00002
Please refer to the end of the specification for access instructions.

LENGTHY TABLE REFERENCED HERE
US20070020623A1-20070125-T00003
Please refer to the end of the specification for access instructions.

LENGTHY TABLE REFERENCED HERE
US20070020623A1-20070125-T00004
Please refer to the end of the specification for access instructions.

LENGTHY TABLE REFERENCED HERE
US20070020623A1-20070125-T00005
Please refer to the end of the specification for access instructions.

LENGTHY TABLE REFERENCED HERE
US20070020623A1-20070125-T00006
Please refer to the end of the specification for access instructions.

LENGTHY TABLE REFERENCED HERE
US20070020623A1-20070125-T00007
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070020623A1) An electronic copy of the table will also be available from the USPTO upon request and payment of the fee set forth in 37 CFR 1.19(b)(3).

1-30. (canceled)

31. A process for the identification of genes expressed in skin, comprising the steps of:
a) isolating a mixture from human or animal skin, wherein the mixture comprises expressed genetically coded factors;
b) subjecting the mixture isolated in a) to a serial analysis of gene expression, wherein genes expressed in skin are identified and their expression quantified.

32. A process for the identification of genes expressed in skin, comprising the steps of:
a) isolating a first mixture from human or animal skin, wherein the mixture comprises expressed genetically coded factors;
b) isolating a second mixture from tissue other than skin, wherein the mixture comprises expressed genetically coded factors;
c) subjecting the first and second mixtures in a) and b) to a serial analysis of gene expression to obtain gene expression patterns for each mixture; and
d) comparing the gene expression patterns obtained in c), wherein genes expressed differentially in skin compared to tissue other than skin are identified.

33. A process for the determination of the homeostasis of the skin, comprising the steps of:
a) isolating a first mixture from skin, wherein the mixture comprises expressed genetically coded factors comprising one or more of proteins, fragments of proteins, mRNA molecules, or fragments of mRNA molecules;
b) isolating a second mixture from tissue other than skin, wherein the mixture comprises expressed genetically coded factors comprising one or more of proteins, fragments of proteins, mRNA molecules, or fragments of mRNA molecules;
c) subjecting the first and second mixtures in a) and b) to a serial analysis of gene expression, wherein the results identify genes expressed differentially in skin as compared to tissue other than skin;
d) isolating a third mixture from skin, wherein the mixture comprises expressed genetically coded factors comprising one or more of proteins, fragments of proteins, mRNA molecules, or fragments of mRNA molecules;
e) obtaining a gene expression pattern for the third mixture by measuring the genetically coded factors produced by expression of at least one of the genes identified in c) as expressed differentially in skin, as compared to tissue other than skin;
f) comparing the gene expression pattern generated in e) with the results of the serial analysis of gene expression in c), wherein a gene expression pattern is indicative of skin in homeostasis if it contains predominantly genetically coded factors expressed by one or more genes identified in c) as expressed more strongly in skin, as compared to tissue other than skin and wherein a gene expression pattern is indicative of skin in disturbed homeostasis if it contains predominantly genetically coded factors expressed by one or more genes identified in c) as expressed more strongly in tissue other than skin as compared to skin.

34. A process for the determination of homeostasis of the skin, comprising the steps of:
a) isolating a mixture from skin, wherein the mixture comprises expressed genetically coded factors comprising one or more of proteins, fragments of proteins, mRNA molecules, or fragments of mRNA molecules;
b) obtaining a gene expression pattern for the mixture by measuring the expression of genetically coded factors expressed by one or more genes defined by their UniGene Accession Number in Tables 1 to 5, column 7 and in Table 7, column 6;
c) comparing the gene expression pattern obtained in b) with the relative expression frequencies shown in Tables 1 to 5 and 7, columns 3 and 4, and the expression quotients indicated in column 5, wherein the gene expression pattern is indicative of skin in homeostasis if it predominantly contains genetically coded factors expressed by genes which are expressed more strongly in skin than in tissues other than skin, whereas the gene expression pattern is indicative of skin in disturbed homeostasis if it predominantly contains genetically coded factors expressed by genes which are expressed more strongly in tissues other than skin than they are in skin.

35. The process according to claim 34, wherein:
in step b), the gene expression pattern for the mixture is obtained by measuring genetically coded factors expressed by one or more genes defined by their UniGene Accession Number in Tables 1 to 5, column 7 and in Table 7, column 6; and
in step c), the gene expression pattern obtained in b) is compared with the relative expression frequencies shown in Tables 1 to 5 and 7, columns 3 and 4, and the expression quotients indicated in column 5, wherein gene expression pattern is indicative of skin in homeostasis if it predominantly contains genetically coded factors expressed by genes which are expressed at least twice as strongly in skin as in tissues other than skin, whereas the gene expression pattern is indicative skin in disturbed homeostasis if it predominantly contains genetically coded factors expressed by genes which are expressed at least twice as strongly in tissues other than skin as they are in skin.

36. The process according to claim 34, wherein:
in step b), the gene expression pattern for the mixture is obtained by measuring genetically coded factors expressed by one or more genes defined by their UniGene Accession Number in Tables 2 to 5, column 7 and in Table 7, column 6; and
in step c), the gene expression pattern obtained in b) is compared with the relative expression frequencies shown in Tables 2 to 5 and 7, columns 3 and 4, and the expression quotients indicated in column 5, wherein gene expression pattern is indicative of skin in homeostasis if it predominantly contains genetically coded factors expressed by genes which are expressed at least five times as strongly in skin as in tissues that are not skin, whereas the gene expression pattern is indicative skin in disturbed homeostasis if it predominantly contains genetically coded factors expressed by genes which are expressed at least five times as strongly in tissues other than skin as they are in skin.

37. The process according to claim 34, wherein:
in step b), the gene expression pattern for the mixture is obtained by measuring genetically coded factors expressed by one or more genes defined by their UniGene Accession Number in Tables 3 to 5, column 7 and in Table 7, column 6; and
in step c), the gene expression pattern obtained in b) is compared with the relative expression frequencies shown in Tables 3 to 5 and 7, columns 3 and 4, and the expression quotients indicated in column 5, wherein gene expression pattern is indicative of skin in homeostasis if it predominantly contains genetically coded factors expressed by genes which are expressed at least ten times as strongly in skin as in tissues that are not skin, whereas the gene expression pattern is indicative skin in disturbed homeostasis if it predominantly contains genetically coded factors expressed by genes which are expressed at least ten times as strongly in tissues other than skin as they are in skin.

38. The process according to claim 34, wherein:
in step b), the gene expression pattern for the mixture is obtained by measuring genetically coded factors expressed by one or more genes defined by their UniGene Accession Number in Tables 4 and 5, column 7 and in Table 7, column 6; and
in step c), the gene expression pattern obtained in b) is compared with the relative expression frequencies shown in Tables 4 and 5 and 7, columns 3 and 4, and the expression quotients indicated in column 5, wherein gene expression pattern is indicative of skin in homeostasis if it predominantly contains genetically coded factors expressed by genes which are expressed at least twenty times as strongly in skin as in tissues that are not skin, whereas the gene expression pattern is indicative skin in disturbed homeostasis if it predominantly contains genetically coded factors expressed by genes which are expressed at least twenty times as strongly in tissues other than skin as they are in skin.

39. The process according to claim 34, wherein:
in step b), the gene expression pattern for the mixture is obtained by measuring genetically coded factors expressed by one or more genes defined by their UniGene Accession Number in Table 5, column 7; and
in step c), the gene expression pattern obtained in b) is compared with the relative expression frequencies shown in Table 5, columns 3 and 4, and the expression quotients indicated in column 5, wherein gene expression pattern is indicative of skin in homeostasis if it predominantly contains genetically coded factors expressed by genes which are expressed at least 100 times as strongly in skin as in tissues that are not skin, whereas the gene expression pattern is indicative skin in disturbed homeostasis if it predominantly contains genetically coded factors expressed by genes which are expressed at least 100 times as strongly in tissues other than skin as they are in skin.

40. A process for the determination of homeostasis of the skin, comprising the steps of:
a) isolating a mixture from skin comprising expressed genetically coded factors comprising one or more of proteins, mRNA molecules, fragments of proteins or fragments of mRNA molecules;
b) quantifying the expression of at least two of the expressed genetically coded factors isolated in a)
c) calculating the expression ratios of at least two of the expressed genetically coded factors whose expression was quantified in b);
d) comparing the expression ratios calculated in c) to the expression ratios shown in Table 6, column 3 and in Tables 1 to 5, column 4, wherein expression ratios calculated in c) corresponding to skin in Table 6, column 3 and in Tables 1 to 5, column 4 are indicative of skin in homeostasis, expression ratios calculated in c) that do not correspond to the expression ratios for skin shown in Table 6, column 3 and in Tables 1 to 5, column 4 are indicative of skin in disturbed homeostasis.

41. A combination comprising a plurality of genes which are differentially expressed in skin in compared to other tissues.

42. The combination of claim 41, wherein the plurality of genes is selected from the genes identified by their UniGene Accession Number in Tables 1 to 5, column 7 and in Table 7, column 6.

43. A combination comprising two or more polynucleotides differentially expressed in skin versus tissue that is not skin, wherein the polynucleotides comprise sequences selected from the group consisting of SEQ ID NOs: 1-7499 and sequences complementary to SEQ ID NOs:1-7499, or fragments thereof.

44. A composition of matter comprising two or more probes for detecting expression of genes differentially expressed in skin compared to other tissues, wherein the probes comprise one or more of oligonucleotides or polynucleotides that specifically hybridize to two or more nucleic acid molecules or fragments thereof that are defined by their UniGene Accession Number in Tables 1 to 5, column 7 and in Table 7, column 6.

45. A composition of matter comprising two or more probes for detecting expression of genes differentially expressed in skin compared to other tissues, wherein the probes comprise polypeptide binding agents that specifically bind to polypeptides or fragments thereof produced by expression of two or more nucleic acid molecules that are defined by their UniGene Accession Number in Tables 1 to 5, column 7 and in Table 7, column 6.

46. The process of claim 34 wherein the mixture is isolated from a skin sample.

47. The process of claim 34 wherein the mixture is isolated from a whole skin sample.

48. The process of claim 34 wherein the mixture is isolated from an epidermis sample.

49. The process of claim 34 wherein the mixture is obtained by microdialysis.

50. The process of claim 34 wherein the gene expression pattern is obtained by measuring the expression of one or more mRNA molecules or fragments of mRNA molecules using one or more methods selected from the group consisting of: northern blots, reverse transcriptase polymerase chain reaction (RT-PCR), RNase protection experiments, dot blots, cDNA sequencing, clone hybridization, differential display, subtractive hybridization, cDNA fragment fingerprinting, total gene expression analysis (TOGA), serial analysis of gene expression (SAGE), and the use of nucleic acid chips.

51. The process of claim 34 wherein the gene expression pattern is obtained by measuring the expression of one or more proteins or fragments of proteins by one or more methods selected from the group consisting of: one- or two-dimensional gel electrophoresis, affinity chromatography, protein/protein complexing in solution, mass spectrometry, Matrix Assisted Laser Desorption Ionization (MALDI), and the use of protein chips.

52. The process of claim 34 wherein 1 to about 5,000 expressed genetically coded factors identified by their UniGene Accession Number in Tables 1 to 5, column 7 and in Table 7, column 6 are quantified in step b).

53. The process of claim 34 wherein 1 to about 1,000 expressed genetically coded factors identified by their UniGene Accession Number in Tables 1 to 5, column 7 and in Table 7, column 6 are quantified in step b).

54. The process of claim 34 wherein about 10 to about 500 expressed genetically coded factors identified by their UniGene Accession Number in Tables 1 to 5, column 7 and in Table 7, column 6 are quantified in step b).

55. The process of claim 34 wherein about 10 to about 250 expressed genetically coded factors identified by their UniGene Accession Number in Tables 1 to 5, column 7 and in Table 7, column 6 are quantified in step b).

56. The process of claim 34 wherein about 10 to about 100 expressed genetically coded factors identified by their UniGene Accession Number in Tables 1 to 5, column 7 and in Table 7, column 6 are quantified in step b).

57. The process of claim 34 wherein about 10 to about 50 expressed genetically coded factors identified by their UniGene Accession Number in Tables 1 to 5, column 7 and in Table 7, column 6 are quantified in step b).

58. A test kit for the determination of stress or ageing of the skin comprising means for carrying out the process of claim 34.

59. A biochip for the determination of stress or ageing of the skin comprising a support and probes which are capable of binding specifically to at least one of the proteins, fragments of proteins, mRNA molecules, or fragments of mRNA molecules which are expressed by genes which are defined by their UniGene Accession Number in Tables 1 to 5, column 7 and in Table 7; wherein the probes are immobilized on the support.

60. The biochip of claim 59 comprising 1 to about 5,000 different probes.

61. The biochip of claim 59 comprising 1 to about 1,000 different probes.

62. The biochip of claim 59 comprising about 10 to about 500 different probes.

63. The biochip of claim 59 comprising about 10 to about 250 different probes.

64. The biochip of claim 59 comprising about 10 to about 100 different probes.

65. The biochip of claim 59 comprising about 10 to about 50 different probes.

66. The biochip of claim 59 wherein the probes comprise nucleic acid probes.

67. The biochip of claim 66 wherein the probes comprise RNA probes.

68. The biochip of claim 66 wherein the probes comprise PNA probes.

69. The biochip of claim 66 wherein the probes comprise DNA probes.

70. The biochip of claim 59 comprising probes having a length of about 10 to about 1,000 nucleotides.

71. The biochip of claim 59 comprising probes having a length of about 10 to about 800 nucleotides.

72. The biochip of claim 59 comprising probes having a length of about 100 to about 600 nucleotides.

73. The biochip of claim 59 comprising probes having a length of about 200 to about 400 nucleotides.

74. The biochip of claim 59 wherein the probes comprise peptide or protein probes.

75. The biochip of claim 74 wherein the probes comprise antibody probes.

76. A process for identifying a substance effective for maintaining or promoting the homeostasis of the skin or for the treatment of pathological conditions of the skin, comprising the steps of:
a) determining the homeostasis of the skin using the process of claim 34,
b) applying at least one substance to the skin,
c) re-determining the homeostasis of the skin by the process of claim 34,
d) identifying effective substances by comparing the results from a) and c.

77. The process of claim 76 wherein the pathological conditions of the skin comprise one or more of neurodermatitis, sunburn, psoriasis, scleroderma, ichtyosis, atopic dermatitis, acne, seborrhoea, lupus erythematodes, roseacea, melanoma, basalioma, skin carcinoma, and skin sarcoma.

78. A screening method for identifying substances effective in treating a pathological condition of the skin comprising the steps of:
a) determining the expression pattern of the skin by assaying a skin sample for the expression of at least one of the proteins, fragments of proteins, mRNA molecules, or fragments of mRNA molecules using the biochip of claim 59,
b) applying a test substance to the skin;
c) re-determining the expression pattern of the skin by assaying a skin sample for the expression of at least one of the proteins, fragments of proteins, mRNA molecules, or fragments of mRNA molecules using the biochip of claim 59; and
d) identifying effective substances by comparing the results from a) and c).

79. A process for determining the effectiveness of a substance in treating a pathological condition of the skin comprising the steps of:
a) contacting a test skin sample with a test substance;
a) obtaining from the test skin sample an expression pattern of at least one of the genes which are identified by their UniGene Accession Number in Tables 1 to 5, column 7 and in Table 7, column 6; and
c) comparing the gene expression pattern obtained in b) with the pattern of a standard skin sample not contacted with the test substance, wherein a difference in the gene expression pattern between the test sample and the standard sample is indicative of the effectiveness of the test substance.

80. The process of claim 79 wherein the pathological condition of the skin comprises one or more of: neurodermatitis, sunburn, psoriasis, scleroderma, ichtyosis, atopic dermatitis, acne, seborrhoea, lupus erythematodes, roseacea, melanoma, basalioma, skin carcinoma, and skin sarcoma.

81. A process for the production of a cosmetic or pharmaceutical preparation for maintaining or promoting homeostasis of the skin comprising the steps of:
a) identifying at least one effective substance by the process of claim 79; and
b) mixing the substance with additional ingredients comprising suitable carriers.
